(19) United States
(12) Patent Application Publication
(10) Pub. No.: US 2019/0059894 A1
(43) Pub. Date: Feb. 28, 2019

(54) MEDICAL STAPLER

(71) Applicant: OLYMPUS CORPORATION, Tokyo (JP)

(72) Inventors: Yoshiyuki KUMADA, Tokyo (JP); Hiroshi OKABE, Tokyo (JP); Keisuke TSURIMOTO, Tokyo (JP); Kayuri KIMURA, Saitama (JP); Masayuki KOBAYASHI, Tokyo (JP)

(73) Assignee: OLYMPUS CORPORATION, Tokyo (JP)

(21) Appl. No.: 16/170,139

(22) Filed: Oct. 25, 2018

Related U.S. Application Data
(63) Continuation of application No. PCT/JP2016/064964, filed on May 20, 2016.

(51) Int. Cl. A61B 17/072; A61B 17/28

(52) U.S. Cl. CPC A61B 17/07207; A61B 17/2804; A61B 2017/07228; A61B 2017/07271; A61B 2017/07264

(57) ABSTRACT

A medical stapler includes a first and a second grippers that are provided so as to be capable of opening/closing with respect to each other, and that can grip tissue therebetween. The first gripper includes a first contact surface being made to come into firm contact with one side of the tissue, and an ejection hole that is provided in the first contact surface and from which a staple is ejected. The second gripper includes a second contact surface being made to come into firm contact with the other side of the tissue, and an anvil portion provided in the second contact surface and having a curved surface that causes a needle portion of the staple to be bent. The first and second grippers are provided so as to allow relative positions thereof to be adjusted in a tangential direction of the curved surface.

### CROSS-REFERENCE TO RELATED APPLICATIONS

This is a continuation of International Application PCT/JP2016/064964 which is hereby incorporated by reference herein in its entirety.

### TECHNICAL FIELD

The present invention relates to a medical stapler.

### BACKGROUND ART

There is a known medical stapler that can cope with tissue having different thicknesses by adjusting bending positions of a symmetrically shaped staple having two piercing needle portions that extend parallel to each other (for example, see Patent Literature 1).

In addition, there is a known medical stapler for suturing tissue by using asymmetrically shaped staples (for example, see Patent Literature 2).

### CITATION LIST

### Patent Literature

{PTL 1} Japanese Translation of PCT International Application, Publication No. 2013-537066
{PTL 2} Publication of U.S. Pat. No. 8,225,980

### SUMMARY OF INVENTION

An aspect of the present invention is a medical stapler including: a first gripper and a second gripper that are provided so as to be capable of opening/closing with respect to each other, and that can grip tissue therebetween in a closed state, wherein the first gripper includes a first contact surface that is made to come into firm contact with one side of the tissue in the closed state, and an ejection hole that is provided in the first contact surface and from which a staple is ejected, wherein the second gripper includes a second contact surface that is made to come into firm contact with the other side of the tissue in the closed state, and an anvil portion that is provided in the second contact surface and that has a curved surface that causes a needle portion of the staple ejected from the ejection hole to be bent in a predetermined direction, and wherein the first gripper and the second gripper are provided so as to allow relative positions thereof to be adjusted in a tangential direction of the curved surface.

### BRIEF DESCRIPTION OF DRAWINGS

FIG. 1 is a longitudinal cross-sectional view showing a medical stapler according to an embodiment of the present invention.

FIG. 2 is a longitudinal cross-sectional view showing an open state in the case in which relatively thin tissue is sutured by using the medical stapler in FIG. 1.

FIG. 3 is a longitudinal cross-sectional view showing a closed state of the medical stapler in FIG. 1 gripping the tissue in the case in FIG. 2.

FIG. 4 is a longitudinal cross-sectional view showing a state in which a staple is ejected from the state in FIG. 3 and is made to pass through the tissue.

FIG. 5 is a longitudinal cross-sectional view showing the relationship between the staple and an anvil portion in the case in FIG. 2.

FIG. 6 is a longitudinal cross-sectional view showing a state in which a needle portion is bent as a result of the staple being further ejected from the state in FIG. 5.

FIG. 7 is a longitudinal cross-sectional view showing the open state in the case in which relatively thick tissue is sutured by using the medical staple in FIG. 1.

FIG. 8 is a longitudinal cross-sectional view showing the closed state of the medical staple in FIG. 1 gripping the tissue in the case in FIG. 7.

FIG. 9 is a longitudinal cross-sectional view showing the relationship between the staple and the anvil portion in the case in FIG. 7.

FIG. 10 is a longitudinal cross-sectional view showing a state in which the needle portion is bent as a result of the staple being further ejected from the state in FIG. 9.

FIG. 11 is a longitudinal cross-sectional view showing a modification of the medical staple in FIG. 1 and the case in which the staple is ejected by being rotated.

FIG. 12 is a front view showing a first modification of the staple used in the medical staple in FIG. 1.

FIG. 13 is a front view of a second modification of the staple used in the medical stapler in FIG. 1.

FIG. 14 is a diagram showing another modification of the medical stapler in FIG. 1.

### DESCRIPTION OF EMBODIMENT

A medical staple 1 according to an embodiment of the present invention will be described below with reference to the drawings.

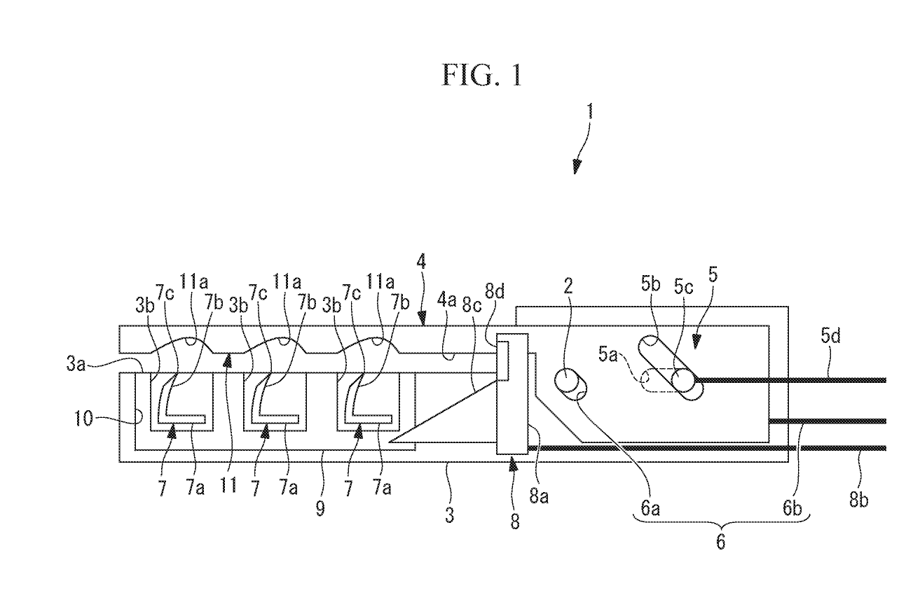

As shown in FIG. 1, the medical stapler 1 according to this embodiment includes a first gripper (first jaw) 3 and a second gripper (second jaw) 4, a pivoting mechanism 5, a shifting mechanism 6, and an ejecting/cutting mechanism 8. The first gripper 3 and the second gripper 4 are provided so as to be pivotable with respect to each other about a pivoting shaft 2. The pivoting mechanism 5 causes the second gripper 4 to be pivoted about the pivoting shaft 2 with respect to the first gripper 3. The shifting mechanism 6 shifts the second gripper 4 with respect to the first gripper 3. The ejecting/cutting mechanism 8 ejects a staple 7 and cuts tissue A.

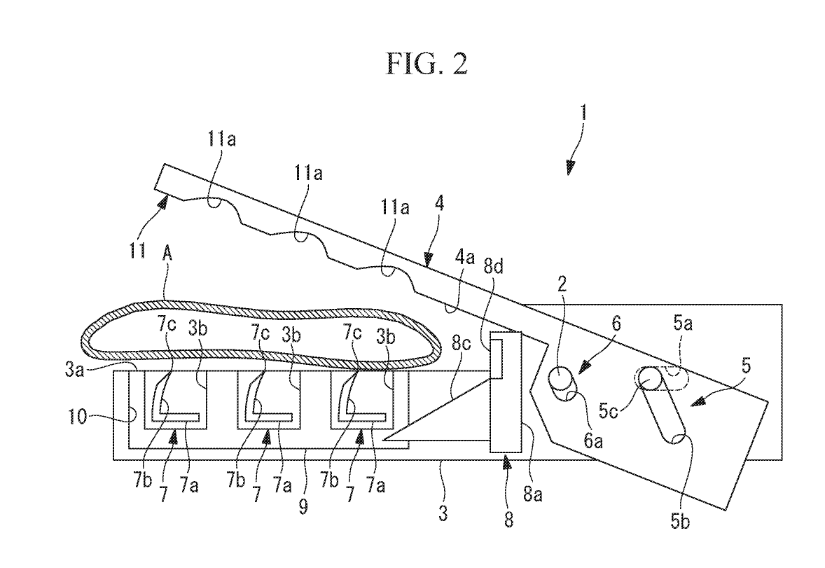

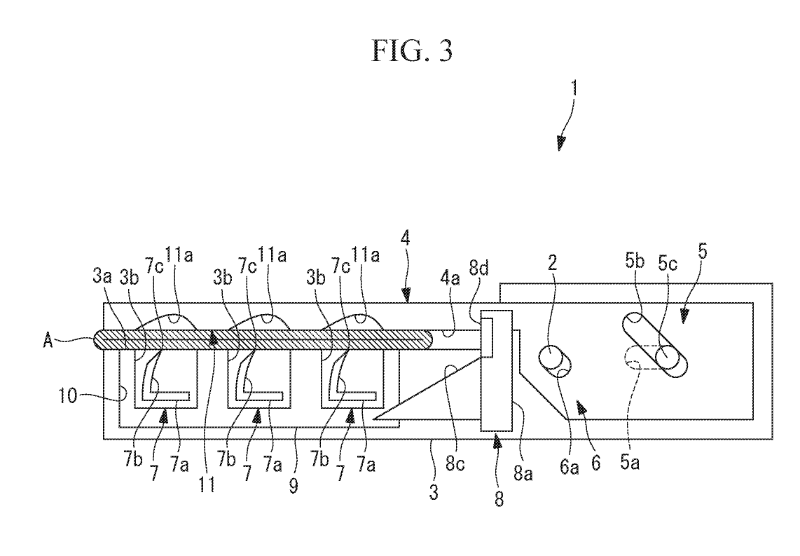

The first gripper 3 and the second gripper 4 are configured so as to be moved relative to each other between an open state shown in FIG. 2 and a closed state shown in FIG. 3, as a result of the pivoting mechanism 5 causing the second gripper 4 to be pivoted about the pivoting shaft 2 with respect to the first gripper 3. In the open state, the first gripper 3 and the second gripper 4 are separated from each other. In the closed state, the first gripper 3 and the second gripper 4 are brought close to each other with the tissue A being gripped therebetween.

The first gripper 3 is provided with a holder 10 that can accommodate a staple cassette 9 in which a plurality of staples 7 are accommodated by being arrayed in two rows (only one row is shown in the figure), for example, at a gripping surface (first contact surface 3a) in the first gripper 3 with which the tissue A is gripped. The first gripper 3 is provided with: the first contact surface 3a that is made to come into firm contact with a surface of the tissue A sandwiched between the first gripper 3 and second gripper 4, in the state in which the staple cassette 9 is accommodated in the holder 10; and ejection holes 3b that are provided in the first contact surface 3a and from which the staples 7 are ejected.

As shown in FIG. 5, the staples 7 have, for example, an asymmetrical shape including a straight base portion 7a and a single needle portion 7b that extends from one end of the base portion 7a in a direction orthogonal to the longitudinal direction of the base portion 7a. The needle portion 7b is formed in a curved shape that is smoothly bent, in a plane including the base portion 7a, in a direction in which the needle portion 7b is brought close the other end of the base portion 7a with an increase in the distance from the base portion 7a. A needle tip 7c of the needle portion 7b is formed in a sharp shape that easily pierces the tissue A.

Also, in the staple cassette 9, the plurality of staples 7 are arrayed with spacings therebetween so that, in each row, the base portions 7a of all of the staples 7 are linearly arrayed, and the base portions 7a and the needle portions 7b of all of the staples 7 are disposed in the same plane. The staples 7 in the individual ejection holes 3b are disposed so that the needle portions 7b point toward the side closer to the openings of the ejection holes 3b.

FIGS. 1 to 3 schematically show an example in which three staples 7 are arrayed in each row. By doing so, in the state in which the staple cassette 9 is accommodated in the holder 10, the ejection holes 3b from which the staples 7 are ejected are also disposed by being arrayed in a single row in each row.

The second gripper 4 is provided with: a second contact surface 4a that is brought into firm contact with the other surface of the tissue A being sandwiched between the first gripper 3 and the second gripper 4 when placed in the closed state; and an anvil portion 11 that is provided in the second contact surface 4a and that is provided with a plurality of grooves that cause the individual staples 7 ejected from the ejection holes 3b to be bent in predetermined directions.

The anvil portion 11 is provided with grooves 11a that are disposed at positions at which the grooves 11a face the individual ejection holes 3b provided in the first gripper 3 when placed in the closed state. In the example shown in FIGS. 1 to 3, three grooves 11a are provided, in each row, at the positions at which the three grooves 11a face the three ejection holes 3b.

Each of the grooves 11a has a groove width that allows a single staple 7 to be inserted thereinto, and also has a groove bottom against which the needle tip 7c of the staple 7 ejected from the ejection hole 3b is made to abut. The groove bottom is formed of a concave curved surface that is smoothly bent so that the depth of the groove 11a reaches zero at the two ends thereof in the longitudinal direction and so that the greatest depth is reached at an intermediate position in the longitudinal direction.

In this embodiment, in both of a state in which second gripper 4 is moved toward the proximal end by the shifting mechanism 6 and a state in which the second gripper 4 is moved to the distal end by the shifting mechanism 6 (described below), the needle tips 7c of the needle portions 7b of the staples 7 ejected from the ejection hole 3b are made to abut against the groove bottoms of the grooves 11a in the anvil portion 11 at angles θ that are less than 90°, as shown in FIGS. 5 and 9.

The pivoting mechanism 5 includes a straight first elongated hole 5a, a straight second elongated hole 5b, a slider member 5c, and an open/close driving-force transmitting member 5d. The first elongated hole 5a is provided in the first gripper 3. The second elongated hole 5b is provided in the second gripper 4 and is inclined so as to intersect the first elongated hole 5a. The slider member 5c is disposed at the position at which the first elongated hole 5a and the second elongated hole 5b intersect so as to pass through the elongated holes 5a and 5b. The open/close driving-force transmitting member 5d pulls and pushes the slider member 5c in a direction along the first elongated hole 5a.

As a result of the open/close driving-force transmitting member 5d pressing the slider member 5c toward the distal end of the first gripper 3, the slider member 5c is moved along the first elongated hole 5a and the second elongated hole 5b, thereby changing the position at which the first elongated hole 5a and the second elongated hole 5b intersect. By doing so, the second gripper 4 is pivoted about the pivoting shaft 2 with respect to the first gripper 3 so as to approach the open state.

Conversely, as a result of the open/close driving-force transmitting member 5d pulling the slider member 5c toward the proximal end of the first gripper 3, the slider member 5c is moved along the first elongated hole 5a and the second elongated hole 5b, thereby pivoting the second gripper 4 about the pivoting shaft 2 with respect to the first gripper 3 so as to approach the closed state.

The shifting mechanism 6 is provided with: a third elongated hole 6a that is provided in the second gripper 4 and through which the pivoting shaft 2 is made to pass; and a shifting-force transmitting member 6b that moves, with respect to the first gripper 3, the second gripper 4 in the length direction of the grooves 11a of the anvil portion 11 provided in the second gripper 4. The third elongated hole 6a is provided so as to be substantially parallel to the second elongated hole 5b.

By doing so, as a result of the second gripper 4 being pressed toward the distal end via the shifting-force transmitting member 6b, the pivoting shaft 2 slides inside the third elongated hole 6a, and thus, the second gripper 4 is disposed at a first position at which the second gripper 4 is shifted with respect to the first gripper 3 in a direction in which a distance therebetween at the distal end and a distance between the first contact surface 3a and the second contact surface 4a increase.

On the other hand, as a result of the second gripper 4 being pulled toward the proximal end via the shifting-force transmitting member 6b, the pivoting shaft 2 slides inside the third elongated hole 6a, and thus, the second gripper 4 is disposed at a second position at which the second gripper 4 is shifted with respect to the first gripper 3 in a direction in which a distance therebetween at the proximal end and a distance between the first contact surface 3a and the second contact surface 4a decrease.

The ejecting/cutting mechanism 8 is provided with: a slider 8a that is provided so as to be linearly movable, inside the holder 10 of the first gripper 3, in a direction in which the staples 7 are arrayed in the staple cassette 9; and a shaft 8b that applies a pressing force to the slider 8a. A slope 8c and a cutter 8d are secured to the slider 8a. The slope 8c causes an ejection force in a direction in which the staples 7 are ejected from the ejection holes 3b to act on the base portions 7a of the staples 7. The cutter 8d is disposed in a gap between the first contact surface 3a and the second contact surface 4a and cuts, as a result of the movement of the slider 8a, the tissue A being gripped between the first contact surface 3a and the second contact surface 4a.

As a result of pressing the slider 8a out toward the distal end by applying the pressing force to the shaft 8b, the base portion 7a of the staple 7 on the extreme proximal-end side is moved up onto the slope 8c, and receives the ejection force from the slope 8c, thus being ejected from the ejection hole 3b. Because the staples 7 are disposed so that the needle portions 7b thereof point toward the openings of the ejection holes 3b, the staples 7 ejected from the ejection holes 3b are ejected outward from the ejection holes 3b from the needle tips 7c of the needle portions 7b first, and the needle portions 7b pass through the tissue A being gripped between the first gripper 3 and the second gripper 4 in the thickness direction.

Also, as a result of further pressing the slider 8a toward the distal end, the needle tips 7c of the needle portions 7b of the staples 7 that have passed through the tissue A abut against the groove bottoms of the grooves 11a of the anvil portion 11 of the second gripper 4. Because the needle tips 7c of the staples 7 are bent so as to lean toward the other ends of the base portions 7a, the needle tips 7c receive external forces from the groove bottoms of the anvil portion 11 in the direction in which the bending thereof is increased.

Then, as a result of the staples 7 being further ejected, the needle tips 7c of the needle portions 7b are moved by being slid along the groove bottoms of the anvil portion 11, are folded back by being bent by a large amount, and are made to pierce the tissue A again. By doing so, the needle portions 7b of the staples 7 are bent so as to wrap around the tissue A, and thus, it is possible to appropriately suture the tissue A.

In addition, the edge of the cutter 8d is disposed so as to point toward the distal end of the first gripper 3, and is configured so as to cut, in the vicinity of the positions sutured by the staples 7, the tissue A that has been sutured by the staples 7 as a result of the staples 7 being ejected by moving the slider 8a toward the distal end.

The operation of the thus-configured medical stapler 1 according to this embodiment will be described below.

In order to suture and cut relatively thin tissue A by using the medical stapler 1 according to this embodiment, first, the second gripper 4 is pulled toward the proximal end of the first gripper 3 via the shifting-force transmitting member 6b of the shifting mechanism 6. By doing so, the third elongated hole 6a is moved with respect to the pivoting shaft 2, and the second gripper 4 in which the third elongated hole 6a is provided is moved toward the proximal end with respect to the first gripper 3 and in a direction in which the second contact surface 4a is brought closer to the first contact surface 3a, as shown in FIG. 1.

Next, as a result of applying a driving force that causes the slider member 5c to be moved toward the distal end of the first gripper 3 via the open/close driving-force transmitting member 5d of the pivoting mechanism 5, the slider member 5c is moved toward the distal end along the first elongated hole 5a provided in the first gripper 3. Because the slider member 5c passes through the second elongated hole 5b provided in the second gripper 4, the second elongated hole 5b is moved so as to change the pass-through position in the second elongated hole 5b. By doing so, as shown in FIG. 2, the second gripper 4 in which the second elongated hole 5b is provided is pivoted about the pivoting shaft 2 with respect to the first gripper 3, thus achieving the open state in which the first gripper 3 and the second gripper 4 are separated from each other.

In this state, as shown in FIG. 2, the tissue A to be sutured is disposed between the first contact surface 3a of the first gripper 3 and the second contact surface 4a of the second gripper 4, and a driving force that causes the slider member 5c to be moved toward the proximal end of the first gripper 3 is applied via the open/close driving-force transmitting member 5d of the pivoting mechanism 5. By doing so, the second gripper 4 is pivoted about the pivoting shaft 2 with respect to the first gripper 3, thereby achieving the closed state in which the two components are closed with respect to each other, as shown in FIG. 3, and thus, the tissue A is gripped between the first contact surface 3a of the first gripper 3 and the second contact surface 4a of the second gripper 4.

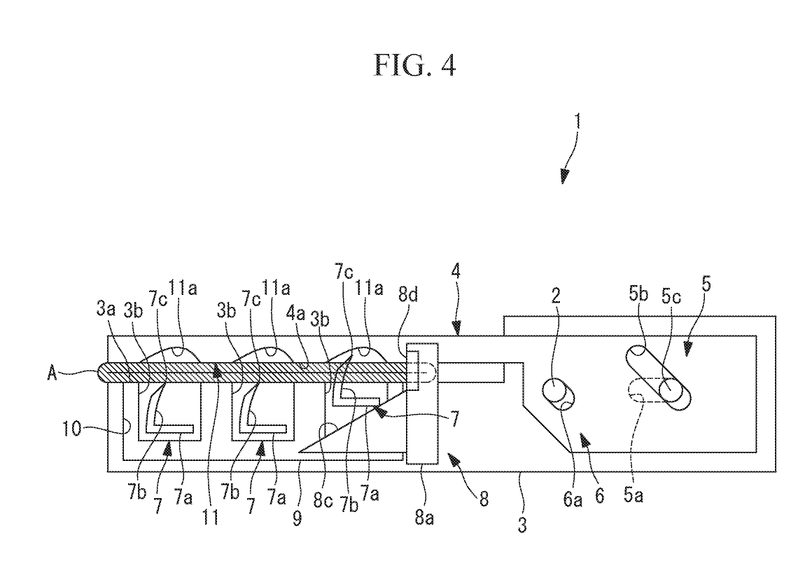

Next, the pressing force is applied to the shaft 8b of the ejecting/cutting mechanism 8. By doing so, the slider 8a is pushed out toward the distal end, as shown in FIG. 4, and, starting from the staples 7 on the extreme proximal-end side, the base portions 7a are made to move up onto the slope 8c, and thus, the staples 7 are ejected from the ejection holes 3b. Because the needle tips 7c of the needle portions 7b are disposed pointing toward the openings of the ejection holes 3b, as a result of the staples 7 being ejected from the ejection holes 3b, the needle portions 7b pass through the tissue A being gripped between the first gripper 3 and the second gripper 4 in the thickness direction of the tissue A.

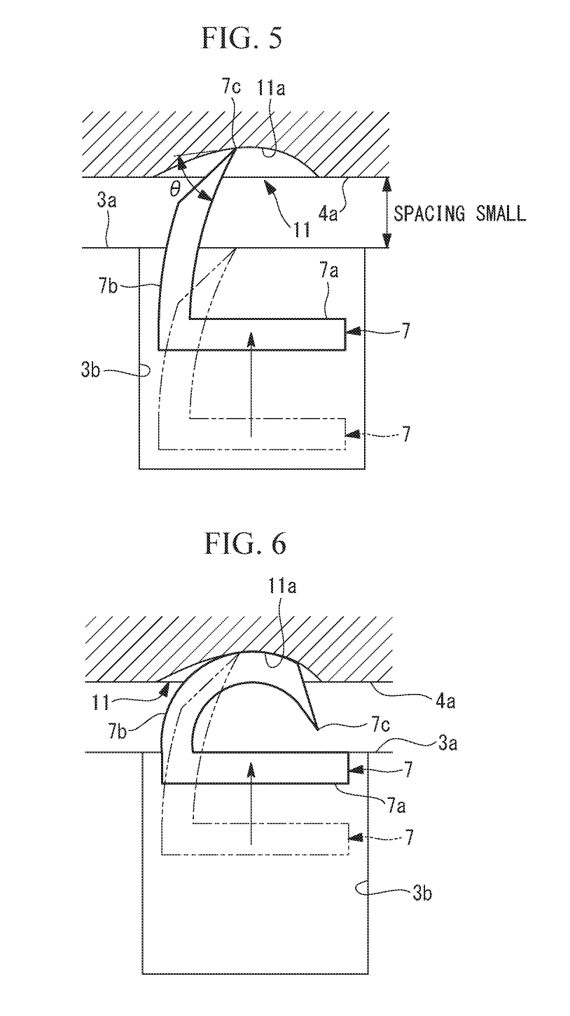

Because the grooves 11a are provided in the second contact surface 4a, which is in contact with the other surface of the tissue A, at the positions opposite from the ejection holes 3b on the other side of the tissue A, the needle portions 7b that have passed through the tissue A are inserted into the grooves 11a, as shown in FIG. 5, and the needle tips 7c abut against the groove bottoms. Because the needle portions 7b are bent toward the base portions 7a and the needle tips 7c abut against the groove bottoms by forming angles θ that are less than 90°, the needle portions 7b receive, from the groove bottoms, forces that cause the needle portions 7b to be bent in one direction. Thus, the needle portions 7b are guided by the shapes of the grove bottoms along the curves thereof, and, as shown in FIG. 6, the needle portions 7b are bent so as to be folded back toward the tissue A, thus piercing the tissue A again. By doing so, it is possible to firmly suture the tissue A with the staples 7.

Also, in association with the motions of ejecting the staples 7, the cutter 8d provided in the slider 8a cuts the tissue A in the proximity of the portions thereof sutured by the staples 7. By doing so, suturing and cutting of the tissue A are repeated by performing the manipulation with which the shaft 8b is pushed out toward the distal end, and thus, it is possible to separate the tissue A in the state in which the tissue A has been appropriately sutured by the staples 7.

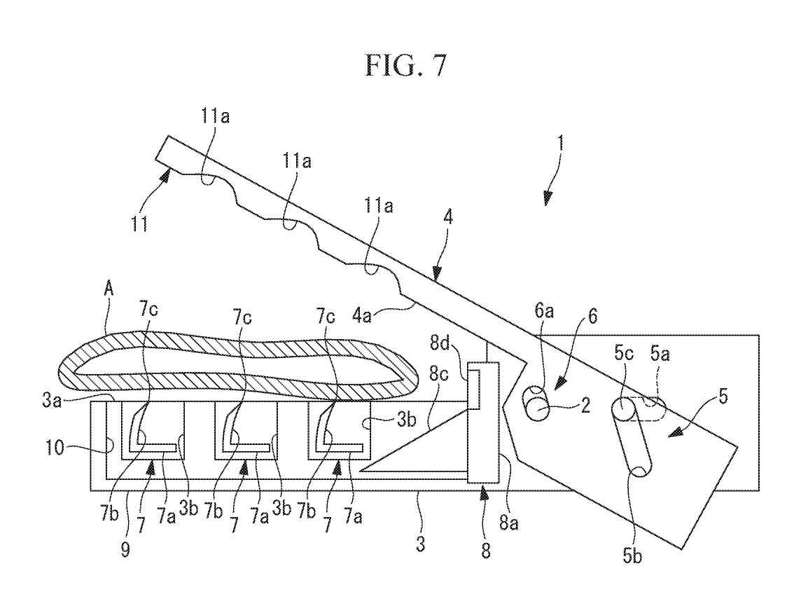

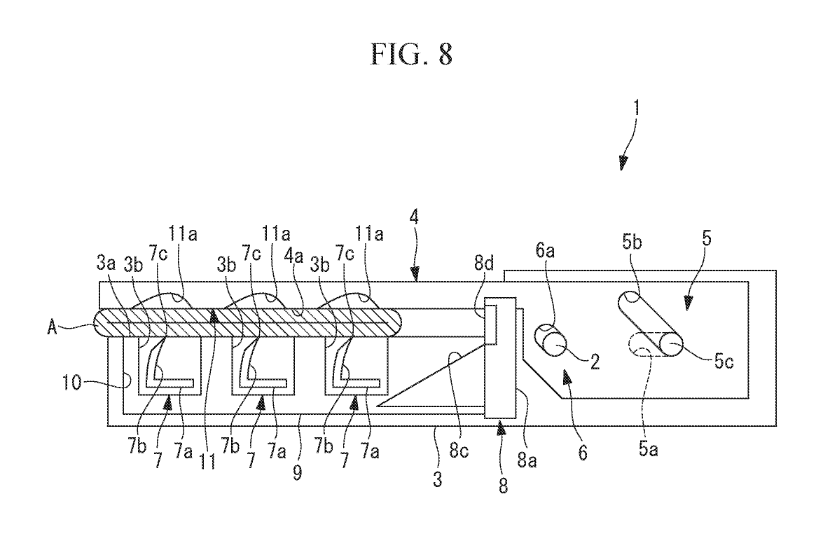

Next, in the case in which relatively thick tissue A is sutured and cut by using the medical stapler 1 according to this embodiment, the second gripper 4 is pushed out toward the distal end of the first gripper 3 via the shifting-force transmitting member 6b of the shifting mechanism 6. By doing so, as shown in FIGS. 7 and 8, the third elongated hole 6a is moved with respect to the pivoting shaft 2, and the second gripper 4 in which the third elongated hole 6a is provided is moved toward the distal end with respect to the first gripper 3 and in a direction in which the second contact surface 4a is separated from the first contact surface 3a.

In this state, the pivoting mechanism 5 is operated, thereby placing the second gripper 4 in the open state with respect to the first gripper 3 and the tissue A is inserted therebetween, as shown in FIG. 7, and the second gripper 4 is placed in the closed state with respect to the first gripper 3, thus gripping the tissue A, as shown in FIG. 8. Then, the ejecting/cutting mechanism 8 is operated, thereby executing suturing of the tissue A by ejecting the staples 7 and cutting of the tissue A by moving the cutter 8d.

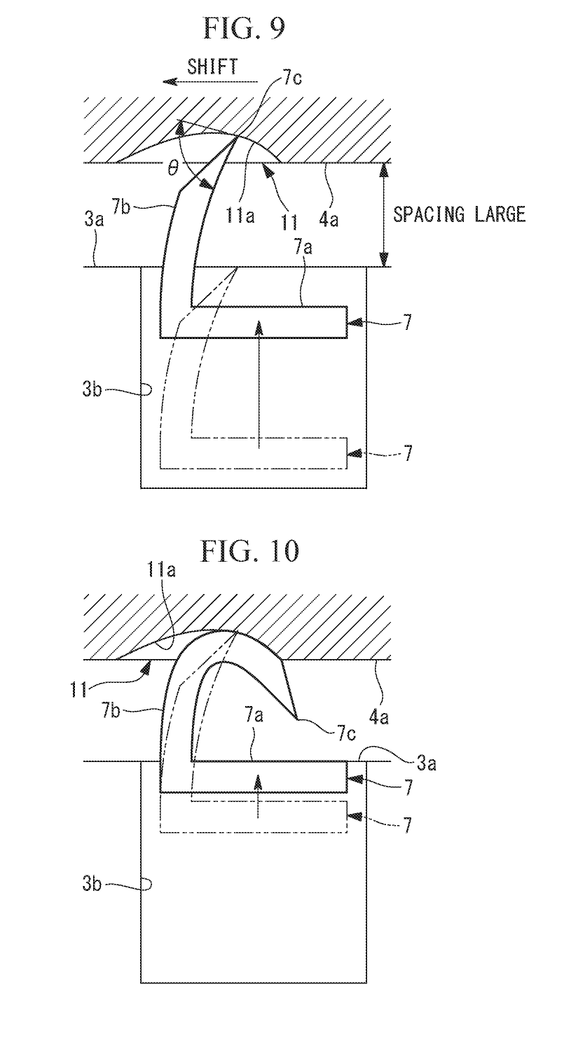

In this case, with the medical stapler 1 according to this embodiment, when suturing the relatively thick tissue A, the positions of the grooves 11a facing the ejection holes 3b for the stapes 7 are shifted toward the distal end, as compared to the case in which the relatively thin tissue A is sutured, as shown in FIGS. 8 to 10. Although, in this case also, the needle tips 7c of the needle portions 7b of the staples 7 abut against the groove bottoms and are made to slide therealong, thereby being bent so as to conform to the curved-shape of the groove bottoms, because the positions at which the groove bottoms are at the deepest are shifted toward the distal end, the needle portions 7b start to be folded back sooner than when suturing the relatively thin tissue A.

In other words, in the case in which the tissue A to be sutured is thick, because the distance between the first contact surface 3a and the second contact surface 4a is greater as compared to the case in which the thin tissue A is sutured, the timing at which the needle tips 7c come into contact with the groove bottoms is delayed if the same staples 7 as those used when suturing the thin tissue A are ejected. With the medical stapler 1 according to this embodiment, with respect to the thick tissue A, the amount of time that passes before the needle portions 7b start to be folded back after the needle tips 7c abut against the groove bottoms is decreased by shifting the second gripper 4 toward the distal end with respect to the first gripper 3. Accordingly, it is possible to suture the tissue A so as to wrap around the tissue A with the needle portions 7b.

In other words, there is an advantage in that it is possible to appropriately suture the relatively thick tissue A and the relatively thin tissue A by using the staples 7 of a single type. As a result, a user does not need to prepare staples 7 of multiples types having different lengths, and, in addition, he/she does not need to perform the procedure for exchanging the staples 7 in accordance to the thickness of the tissue A to be sutured.

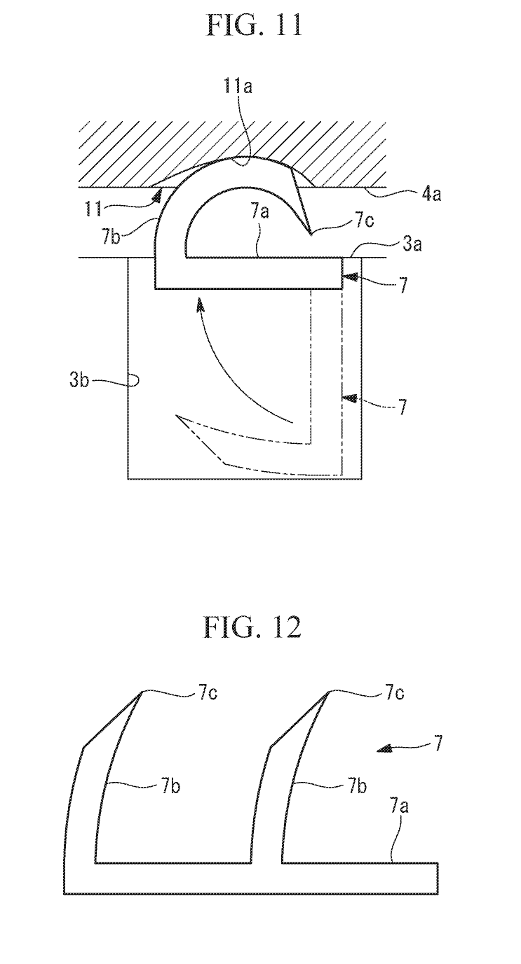

Note that, in the medical stapler 1 according to this embodiment, although the unit employing the system in which the staples 7 are ejected by being translated has been described as an example, there is no limitation thereto, and the present invention may be applied to a unit employing a system in which the staples 7 are ejected by being rotated, as shown in FIG. 11.

In addition, although the staples 7 each having the single needle portion 7b have been described as an example, alternatively, as shown in FIG. 12, an asymmetrical shaped staple 7 having two or more needle portions 7b may be used.

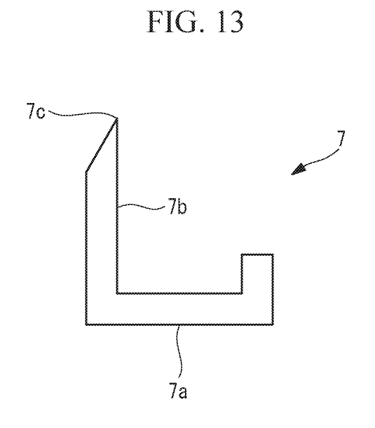

In addition, although the staples 7 having forms in which the needle portions 7b are curvilinearly bent are employed so as to facilitate bending of the needle portions 7b by means of the curved surfaces of the grooves 11a, as shown in FIG. 13, staples 7 having straight needle portions 7b may be employed.

In addition, in this embodiment, although the case in which the manipulations for switching between the two levels, that is, the relatively thin tissue A and the relatively thick tissue A, is manually performed by means of the shifting mechanism 6 has been described as an example, alternatively, the switching may be performed among three or more levels or in a continuous manner in accordance with the thickness of the tissue A.

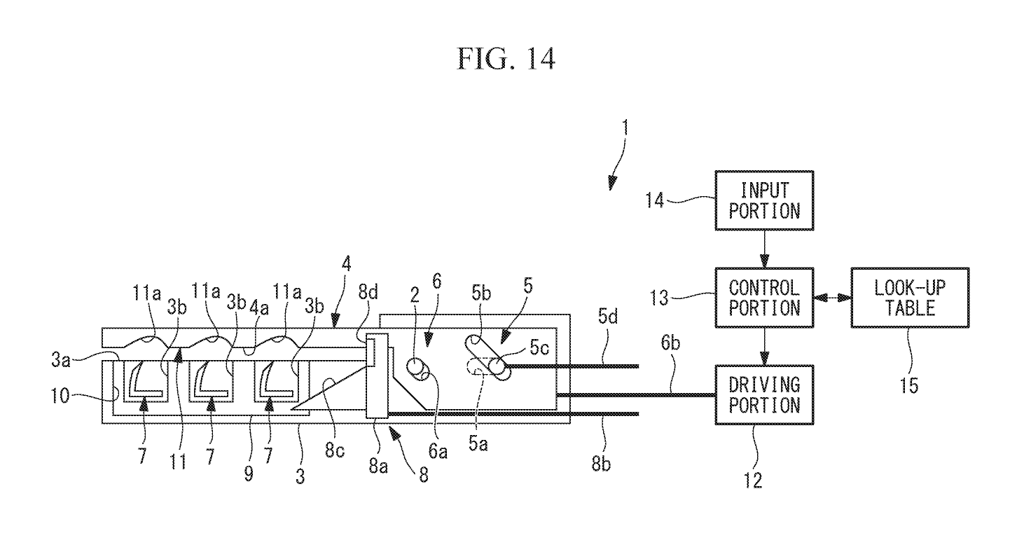

In this case, as shown in FIG. 14, it is preferable that a driving portion 12 that applies a driving force to the shifting-force transmitting member 6b and a control portion 13 that controls the driving portion 12 be included.

As the driving portion 12, it is possible to employ a publically known arbitrary linear motion mechanism, such as a motor and a ball screw or the like (not shown).

The example shown in FIG. 14 is provided with: an input portion 14 with which the user inputs a classification or thickness of the tissue A; and a look-up table 15 that stores, in association with each other, the classification or the thickness of the tissue A and the amounts by which the second gripper 4 is shifted by the shifting mechanism 6.

When the classification or the thickness of the tissue A is input from the input portion 14, the control portion 13 refers to the look-up table 15, reads out the shifting amount of the second gripper 4 corresponding thereto, and controls the driving portion 12 so that the second gripper 4 is shifted by an amount corresponding to the read-out shifting amount.

By doing so, there is an advantage in that it is possible to appropriately suture the tissue A having various thicknesses by means of the staples 7 of a single type by more finely adjusting the amounts by which the second gripper 4 is shifted with respect to the first gripper 3.

Note that, instead of the look-up table 15, a formula may be stored, and the shifting amounts may be calculated on the basis of the thicknesses input from the input portion 14.

In addition, although a case in which the second gripper 4 is shifted with respect to the first gripper 3 has been described as an example, the first gripper 3 may be shifted with respect to the second gripper 4, or both of the grippers 3 and 4 may be shifted.

In addition, although the case in which three staples 7 are arrayed in two rows has been described as an example in order to simplify the illustration and descriptions, the number of the staples 7 in each row and the number of rows may be arbitrary.

### REFERENCE SIGNS LIST

1 medical stapler
3 first gripper
3a first contact surface
3b ejection hole
4 second gripper
4a second contact surface
7 staple
7b needle portion
11 anvil portion
13 control portion
14 input portion
15 look-up table
A tissue

1. A medical stapler comprising:
a first gripper and a second gripper that are provided so as to be capable of opening/closing with respect to each other, and that can grip tissue therebetween in a closed state,
wherein the first gripper includes a first contact surface that is made to come into firm contact with one side of the tissue in the closed state, and an ejection hole that is provided in the first contact surface and from which a staple is ejected,
wherein the second gripper includes a second contact surface that is made to come into firm contact with the other side of the tissue in the closed state, and an anvil portion that is provided in the second contact surface and that has a curved surface that causes a needle portion of the staple ejected from the ejection hole to be bent in a predetermined direction, and
wherein the first gripper and the second gripper are provided so as to allow relative positions thereof to be adjusted in a tangential direction of the curved surface.

2. The medical stapler according to claim 1, wherein the first gripper and the second gripper are provided so as to allow a distance between the first contact surface and the second contact surface in the closed state to be adjusted.

3. The medical stapler according to claim 1, wherein the anvil portion is brought into contact with a distal end of the needle portion of the staple ejected from the ejection hole at an angle that is less than 90°.

4. The medical stapler according to claim 1, further comprising:
an input portion with which a thickness of the tissue is input; and
a control portion that sets an amount by which the relative positions between the first gripper and the second gripper are adjusted in the tangential direction so as to be greater with an increase in the thickness of the tissue input via the input portion.

5. The medical stapler according to claim 1, further comprising:
an input portion with which a classification of the tissue is input;
a look-up table that stores the classification of the tissue and an amount by which the relative positions between the first gripper and the second gripper are adjusted in the tangential direction in association with each other; and
a control portion that causes the first gripper and the second gripper to be moved relative to each other in the tangential direction on the basis of the amount read out from the look-up table on the basis of the classification of the tissue input via the input portion.
